(19) United States
(12) Patent Application Publication
(10) Pub. No.: US 2009/0186356 A1
(43) Pub. Date: Jul. 23, 2009

(54) DEVICE AND SUBSTANCE FOR THE IMMOBILIZATION OF MESENCHYMAL STEM CELLS (MSCs)

(75) Inventors: Hans-Peter Wendel, Balingen (DE); Ketai Guo, Tuebingen (DE); Richard Schaefer, Moessingen (DE)

Correspondence Address: KLARQUIST SPARKMAN, LLP, 121 SW SALMON STREET, SUITE 1600, PORTLAND, OR, 97204, US

(73) Assignee: Eberhard-Karls-Universitaet Tuebingen Universitaetsklinikum

(21) Appl. No.: 12/324,698

(22) Filed: Nov. 26, 2008

Related U.S. Application Data
(63) Continuation of application No. PCT/EP2007/004057, filed on May 8, 2007.

(30) Foreign Application Priority Data
May 26, 2006 (DE) 10 2006 026 191.7

(51) Int. Cl. C12Q 1/68; C12M 1/26; C12N 5/02; C07H 21/04

(52) U.S. Cl. 435/6; 435/309.1; 435/378; 536/23.1

(57) ABSTRACT

The invention relates to a device comprising at least one surface which comes into contact with biological tissue and/or liquid, which is at least partially coated with a substance which mediates the binding of mesenchymal stem cells (MSCs), a method for the binding and/or isolation of MSCs from biological tissue and/or liquid, a nucleic acid molecule which selectively and highly specifically binds to MSCs, the use of the nucleic acid molecule for the binding and/or isolation of MSCs from biological tissue and/or liquid, as well as a method for the production of a device mentioned at the outset.

### CROSS-REFERENCES TO RELATED APPLICATIONS

This application is a continuation application of co-pending international patent application PCT/EP2007/004057 filed on May 8, 2007 and designating the United States, and claims priority of German patent application DE 10 2006 026 191.7 filed on May 26, 2006, which are incorporated herein by reference.

### BACKGROUND OF THE INVENTION

1. Field of the Invention

The present invention relates to a device comprising at least one surface which comes into contact with biological tissue and/or liquid, which is at least partially covered with a substance which mediates the binding of mesenchymal stem cells (MSCs), a method for the binding and/or isolation of MSCs from biological tissue and/or liquid, a nucleic acid molecule which selectively and highly specifically binds to MSCs, the use the nucleic acid molecule for the binding and/or isolation of MSCs from biological tissue and/or liquid, as well as a method for the production of the device mentioned at the outset.

2. Related Prior Art

Devices for the binding of MSCs and methods for the binding and/or isolation of MSCs from biological tissue and/or liquid are generally known in the art.

Stem cells are undifferentiated cells which have the capability to renew themselves and to differentiate into different effector cells. Due to these characteristics they belong to the most promising subjects of the biomedical research and nurture hope that in the future tissues or entire organs, respectively, can be regenerated within the frame of the so-called stem cell therapy.

Embryonic stem cells and adult stem cells can be differentiated from each other according to their origin.

Embryonic stem cells are obtained from the internal cell mass of the blastocyst stadium of a mammalian or a human embryo. They can divide in an unlimited manner and, theoretically, can develop into each cell type of the about 200 kinds of tissues of a human. Obtaining embryonic stem cells from the blastocysts within the frame of stem cell research often results in ethical conflicts since the death of the embryo has to be accepted.

In contrary to this, the research on adult stem cells is ethically absolutely harmless since they are obtained from adult organisms. Adult stem cells so far have been discovered in at least 20 tissues of the human being. Their function is to form replacement cells for the corresponding tissues. In comparison with embryonic stem cells so far they have been considered as having a limited capacity for division and development: It was assumed that adult stem cells of a specific tissue cannot form types of cells of another tissue. Recently, evidence is mounting that also adult stem cells comprise a considerably higher potential of development as so far assumed. In the meantime, many of the experts are of the opinion that also adult stem cells can take several paths of differentiation.

The so-called mesenchymal stem cells (MSCs) also belong to the adult stem cells. MSCs can be found in a multitude of tissues and organs such as the liver, the kidney, the placenta, in the fat tissue, in the cord blood as well as in the bone marrow. In general, MSCs are characterized as multipotent CD29⁺, CD44⁺, DC90⁺, CD11b⁻, CD34⁻ and CD45⁻ progenitor cells which, due to their mesenchymal differentiation potential and their good expansion properties in vitro, constitute an attractive cell population for the use of mesenchymal tissue within the frame of the so-called tissue engineering. MSCs can be differentiated in vitro into cartilage, bones, tendons and fat cells. MSCs, when three-dimensionally cultivated on different carrier substances, have already been used in many small animal and large animal studies to replace mesenchymal tissue. Also a clinical use of MSCs in humans has already been successfully tested in pilot studies.

Up to now, the isolation of MSCs from a biological sample is based on the selection factor of plastic adherence, i.e. on their capability to adhere to specific plastic surfaces, and on media conditions which are characteristical for MSCs, cf. Pittenger et al. (1999), Multilineage potential of adult human mesenchymal stem cells, Science 284, pages 143-147, Reyes (2001), Purification and ex vivo expansion of postnatal human marrow mesodermal progenitor cells, Blood 98, pages 2615-2625.

The disadvantage of this method of the art is the fact that different populations of MSCs are obtained. Consequently, such MSC cells display different biological characteristics, e.g. with regards to their proliferation behavior and their synthesis of matrix; cf. Niemeyer et al. (2003), Vergleich zweier Isolationsverfahren zur Gewinnung humaner mesenchymaler Stammzellen aus Knochenmark [“Comparison of two isolation methods for obtaining human mesenchymal stem cells from bone marrow”], Z. Orthop. 141, page 129. MSCs obtained by plastic adherence are therefore less appropriate for the tissue engineering since here particularly high demands are to be made on the homogeneity of the MSC population and the reproducibility of the obtainment of MSCs.

Another disadvantage of the method of isolation via plastic adherence which is so far performed to obtain MSC relates to the fact that it is very time consuming and takes at least two weeks.

The object underlying the invention is therefore to provide a device and a method for the binding and/or isolation of MSCs from biological tissue and/or liquid, which overcome the before-mentioned disadvantages of the art.

This object is achieved by the device mentioned at the outset, which is coated with aptamers. This object is further achieved by a method which comprises the following steps: (1) providing biological tissue and/or liquid which contain MSCs, (2) contacting said tissue and/or said liquid with a substance which binds to MSCs, (3) incubating for a period of time which allows the binding of the MSCs to said substance, and, if applicable (4) isolating of MSC which are bound to the substance, whereby the substance is an aptamer.

The object underlying the invention is herewith completely achieved. The inventors have surprisingly realized that capture molecules in form of aptamers can be provided which selectively and highly specifically bind to MSCs. This finding was in particular surprising since so far no reliable MSC markers are known. Some years ago STRO-1 was discussed as a promising surface antigen for the identification of MSCs, in the meantime its importance has been qualified; cf. Dennis et al., The STRO-1+ marrow cell population is multipotential, Cells Tissues Organs 170, pages 7-82. Also the antigen W8B2 of unknown identity, which was recently described as a MSCs marker, shows a heterogeneous expression on populations of MSC; cf. Vogel et al. (2003), Heterogenity among human bone marrow-derived mesenchymal stem cells and neuroprogenitor cells, Haematologica 88, pages 126-133.

For this reason, so far neither antibodies nor aptamers are known which selectively and highly specifically bind to MSCs. In this context, the DE 102 58 924 A1 mentions aptamers which should bind to stem cells, however only such aptamers are disclosed which exclusively bind to endothelial progenitor cells (EPCs), however not to MSCs.

From the U.S. Pat. No. 6,287,765 B 1 and WO 03/065881 A2 devices of unknown purpose are known, which comprise nucleic acid molecules of unknown characteristics, by which biological material may be captured.

Furthermore, a large number of devices are described in the art, which are coated with peptides, such as receptors or antibodies, which may capture biological material.

Aptamers are highly affine RNA or DNA oligo- or polynucleotides, respectively, i.e. nucleic acid molecules which, due to their specific three dimensional structure, comprise a high affinity to a target molecule. Usually aptamers comprise a length of up to 100 nucleotides which comprise antigen binding properties comparable to antibody fragments, however, very often are considerably more specific and are remarkably increased in their stability. With their relatively large and flexible surface they can potentially interact with much more target molecules in a highly specific and selective manner. Aptamers, when introduced into an organism, almost have no immunogenic or toxic effects, however, show a rapid clearance.

By means of “SELEX” (Systemic Evolution of Ligands by Exponential Enrichment) large amounts of aptamers of different sequences and secondary structures can be enzymatically produced. In the following such aptamers of this mass comprising a high affinity to a target molecule, such as to MSCs, are identified and amplified. The primary structure of such an aptamer can be elucidated by means of sequencing methods known in the art, so that in the following it can be synthesized in vitro. A model method for obtaining aptamers is e.g. described in the DE 100 19 154 A1, which is incorporated herein by reference.

Devices according to the invention can be used extracorporally or in the form of implants. An extracorporal device which is coated with aptamers according to the invention, can be brought into contact with biological tissue or liquid which is to be analyzed for the presence of MSCs. Furthermore, for the targeted isolation of MSCs such a device can be brought into contact with tissues or liquids, which are known for the presence of MSCs. Examples of such MSC containing biological tissues or liquids are bone marrow, peripheral blood or apheresis blood. After contacting the device with the tissue or the liquid, respectively, a is followed to allow the binding of the MSCs, if present, to the aptamers. After the incubation the device comprising the MSCs bound via the aptamers is separated from the tissue or the liquid. This has the advantage that the bound MSCs do not necessarily have to be separated from the aptamers, since, according to the storage conditions, the aptamers are completely degraded within a short period of time, e.g. within two days.

Against this background, the device can be realized by a simple carrier coated with aptamers, however also by a tube, a pump, an oxygenator, a catheter, a vascular gateway, a storage system for blood components.

A coating with aptamers has also the advantage that it is stable and can be sterilized, resulting in a cost-effective production. On the contrary to this peptides very often lose their activity when sterilized.

According to the invention, biological tissue and/or liquid refers to any biological material of animal or human origin or any liquid, which is to be analyzed for the presence of MSCs. This applies to a tissue formation, a cell suspension or organs, parts of organs or organisms. Examples of biological tissues or liquids are bone marrow tissue, bone marrow cells, cartilage cells, bone cells, fat tissue, fat cells, liver tissue, liver cells, placenta tissue, placenta cells, peripheral blood, cord blood, apheresis blood.

It shall be understood that a binding and/or isolation of MSCs from biological tissue and/or liquid is also possible with such aptamers which are not bound to the surface of the device according to the invention, i.e. which are added to the tissue or the liquid in loose form and, if appropriate, in the following can be re-isolated by methods known in the art. Against this background another subject-matter of the present invention relates to a nucleic acid molecule or an aptamer, respectively, which selectively and highly specifically binds to MSCs, as well as its use for the binding and/or isolation of MSCs. “Selectively” and “highly specifically” means in this connection that the nucleic acid molecule or aptamer, respectively, binds to MSCs in a targeted manner and interactions with other structures do not take place to a large extent or are missing entirely or are within the frame of common cross reactivities.

It is preferred if the aptamer is a nucleic acid molecule which comprises at least one of the sequences SEQ ID NO: 1 to SEQ ID NO: 20 of the enclosed sequence listing.

This measure has the advantage that a primary structure of such an aptamer is already provided which highly specifically and selectively binds to MSCs. The performance of a SELEX method is then not necessarily required. Then the intended aptamer can be directly produced by means of simple and time-saving synthesis methods.

It shall be understood that such a sequence-specific aptamer according to the invention can still bind to MSCs in highly specific and selective manner, if in addition to one of the nucleotide sequences SEQ ID NO: 1 to SEQ ID NO: 20 it comprises at its 5′- or 3′-end, respectively, one or several other nucleotides. The same applies for the case when in the non-functional areas of the aptamer one or several nucleotides are replaced or are absent. The selectivity and specificity of the aptamer of this embodiment is preserved since the replacement or exchange, respectively, occurs outside the so-called “hair pin loops” or “bulks”, which are the functional areas of the aptamer. These areas form the secondary structures which are responsible for the binding to the target structure. If two aptamers correspond to each other in their nucleotide sequences within these functional areas, however differ in their nucleotide sequences in non-functional segments, they can bind to the same target structure. Against this background, this embodiment according to the invention also encompasses such an aptamer which comprises the functional segments of the nucleotide sequences SEQ ID NO: 1 to SEQ ID NO: 20, which however is modified in the non-functional segments by nucleotide substitutions or deletions. Such a modified aptamer is in its capability to bind to MSCs not or not essentially altered.

The sequence specific aptamers in question can also be modified by means of appropriate techniques which protect and prevent them from losing their activity in a biological environment, e.g. due to the a digest by nucleases. Preventive measures which are appropriate for this purpose are sufficiently described in the art and include e.g. LNA (locked nucleic acids) technologies with furanose [see e.g. Wahlestedt et al. (2000), Patent and non toxic antisense oligonucleotides containing locked nucleic acids, Proc. Natl. Acad. Sci. USA 97 (10), pages 5633 bis 5638)], or the Spiegelmer® technology of the company Noxxon, Berlin, Germany.

It is further preferred if the aptamer comprises a detectable and/or selectable marker.

By this measure MSCs can be detected and selected in an especially simple manner. According to the invention, a marker refers to any compound by means of which a localization and identification of the aptamer in vitro, in vivo, or in situ is possible. This applies to color indicators with fluorescent, phosphorescent or chemoluminescent properties, such as fluorescein isothiocyanate (FITC), rhodamine, AMPPD, CSPD, radioactive indicators such as ³²P, ³⁵S, ¹²⁵I, ¹³¹I, ¹⁴C, ³H, non-radioactive indicators such as biotin or digoxigenin, alkalic phosphatase, horseradish peroxidase, etc.

By using a fluorescence labeled aptamer the method according to the invention can be performed within the frame of the established fluorescence activated cell sorting (FACS). By means of FACS the MSCs can be isolated from a cell suspension in a particularly well manner. For doing this, a cell suspension containing MSCs is incubated with fluorescence-labeled aptamers. The aptamers then bind to the MSCs. The cell suspension is then passed through a thin cannula, at its end the jet of the cell suspension is disintegrated into single drops by vibration. If one drop contains an MSC to which an aptamer is bound the fluorescent marker is excited by a laser beam for fluorescence. This fluorescence can be measured by a light detector and can be used for the separation and therefore isolation of the MSC. For this, the bound MSCs are, according to the intensity of fluorescence, electrified by means of an electric impulse and are deflected and sorted correspondingly when passing an electric field.

Selectable markers are e.g. magnetic particles which are preferably very small in the dimension of 50 nm. They can be coupled to the aptamers by means of methods known in the art. Such magnetic aptamers can also be used to isolate MSCs, namely within the context of the so-called magnetic cell sorting (MACS). For this, the magnetic aptamers are added to the cell suspension. After an incubation period the aptamers have been bound to the MSCs. The cell mixture is separated via a column, the ferromagnetic matrix of which consists of metal beads or wires. For this, the column is placed into a homogenous magnetic field, where the MSCs, to which the magnetic aptamers are bound to, are held to the surface of the matrix. The remaining cells and components of the mixture are washed from the column. After removing the magnetic field, the separated MSCs can also be diluted from the matrix. This method enables a rapid separation of MSCs without strong mechanical interferences and with a high degree of concentration, i.e. also a very small population of MSCs can almost be isolated in pure form.

According to a preferred further development, the device according to the invention is an implant.

According to the invention, this refers to a device which is introduced in the human or animal body either for a specific period of time or permanently. This applies to artificial cardiac valves, artificial hip or knee joints, cardiac pacemakers, dental implants, plates and screws, vascular prostheses, conduits, catheters, artificial bladders, which in principle can consist of any polymeric plastics, metals, alloys, textiles, natural materials (chitosan), bacterial cellulose, etc. but also of other stable or degradable materials.

Further, in the vascular surgery very often prostheses, e.g. in the form of stents, are used, which can be made of different plastics or materials. In relation with stents but also with other vascular prostheses or gateways, ports or conduits it can be advantageous if not necessarily all surfaces are coated with the aptamer according to the invention, but only specific faces, such as the internal surface which should come into contact with blood. It can furthermore be intended to cover the surface(s) locally with different aptamers according to the invention, so that different populations of MSC can be bound.

The invention enables in an advantageous manner a colonization of the implants with the body's own MSCs. By this on the one hand it is ensured that on the implant an autologous functional interface is generated, which is no longer recognized by the body as being foreign, and on the other side that the implant takes over the functional physiological properties of the corresponding site of operation or the organ, e.g. as bone substitute, dental implant, etc.

The colonization of the device according to the invention with MSCs can occur intracorporally, extracorporally, but also in a separate bioreactor within which the biological tissue and/or liquid is contained.

The implants can also be realized by so-called patches or foils which are to be coated with MSCs. Such a patch consists e.g. of poly-N-isopropylacryl amide (PIPAAm) as described in Miyahara et al. (2006). Monolayered mesenchymal stem cells repair scarred myocardium after myocardial infarction, Nature Medicine, Online Publication, pages 1 to 7. The authors describe the transplantation of patches colonized with MSCs into a heart impaired by a myocardial infarction, which showed, due to the inserted MSCs, an astonishing well regeneration. However, the authors at first had to colonize the patch with previously isolated MSCs, and only the colonized patches were then implanted into the patients. Due to the present invention a patch coated with aptamers can be directly introduced into the heart where in the following the affinity of the aptamers on its own takes care for the colonization with MSCs. Having patches colonized with MSCs in stock the corresponding complex previous isolation and colonization of the patches with MSCs is no longer required. With this measure a patient in need can be helped more rapidly.

In another embodiment it is preferred if as a surface for the device according to the invention a material is used which is selected from the group consisting of polytetrafluoroethylene, poly-styrene, polyurethane, polyester, polylactide, polyglycolic acid, polysulphone, polypropylene, polyethylene, polycarbonate, polyvinyl chloride, polyvinyl difluoride, polymethyl methacrylate, hypoxylapatite, isopropylacrylamide, texin or copolymers thereof, nylon, silanized glass, ceramics, metals, in particular titanium, or mixtures thereof.

Such materials have been proven in these specific fields, e.g. in the tissue engineering, and are used in different versions. The form of the surface can be chosen in a user-defined manner.

It is preferred if the aptamers are either directly and/or via a linker molecule attached to the surface of the device according to the invention.

“Linker molecule” or “linker” refers to any substance by which an aptamer can be attached to the surface. Aptamers can in principle—like any nucleotides (e.g. after coupling to amino or biotin groups)—be linked via appropriate linker molecules or spacers attached to the surface of the device. Methods for immobilizing oligonucleotides are e.g. described in “Immobilisierung von Oligonucleotiden an aminofunktionalisierte Silizium-Wafer” [“Immobilization of oligonucleotides to aminofunctionalized silicium wafers”] (U. Haker, Chem. Diss., Hamburg, 2000), where inter alia 1,4-phenylene diisothiocyanate is used. In the dissertation “Miniaturisierte Affinitätsanalytik—Ortsaufgelöste Oberflächenmodifikationen, Assays und Detektion” [“Miniaturized affinity analytics—space resolved modifications of surfaces, assays and detection”] (I. Stemmler, Chem. Diss., Tübingen, 1999) and in the publication of Hermanson et al., “Immobilized affinity ligand techniques” (Academic Press, San Diego, 1992) and “Bioconjugate Techniques” (Academic Press, San Diego, 1996) further important covalent methods for the modification of surfaces are presented. For example, as a functional anchor SiO₂, TiO₂, —COOH, HfO₂, —Au, —Ag, N-hydroxysuccinimide, —NH2, epoxide, maleinimide, acidic hydrazide, hydrazide, azide, diazirine, benzophenone, and others, can be used in couplings with different reaction partners.

An appropriate substance for attaching an aptamer to the surface of a device according to the invention is a hydrogel which is marketed by the company Schott, Mainz, Germany, under the designation Nexterion®, to which e.g. amino-modified aptamers can be covalently bound. A coating of the device according to the invention with a hydrogel which is compatible with blood can be of an advantage, e.g. belonging to the group of PEGs or Star PEGs, which e.g. comprise a free carboxy group to which e.g. an amino-modified aptamer can be covalently bound. This measure has the advantage that other blood cells, e.g. thrombocytes or plasma proteins, e.g. fibrinogen, cannot bind to the surface and therefore do not overlay or “clot” the binding sites of the aptamers for MSCs.

Another method for immobilizing oligonucleotides on surfaces is the photo linking. Here, the NH₂ coupled oligonucleotide (aptamer) is at first provided with a so-called photo linker molecule (e.g. anthraquinone) which in the following upon UV activation can photochemically react with a plastic surface and thereby can couple the oligonucleotide covalently to the surface. Kits and substances required to perform this method are commercially obtainable e.g. under the designation AQ-Link™ and DNA Immobilizer™ of the company Exiqon (Vedbaek, Denmark).

It is, however, preferred if the linker molecule is N-succinimidyl-3-(2-pyridyldithio)propionate.

For the substance N-succinimidyl-3-(2-pyridyldithio)propionate it could be demonstrated that it can already be used during the immobilization of a regulator of the complement system on specific surfaces of biomaterials (see Andersson et al. “Binding of a model regulator of complement activation (RCA) to a biomaterial surface: surface-bound factor H inhibits complement activation”, Biomaterials 22: 2435-2443, 2001). By using this linker the biological activity of the regulator was not affected.

In another embodiment the device according to the invention can additionally be coated with growth factors. This embodiment has the advantage that the bound MSCs can be differentiated by means of specific growth factors into the intended direction which results in a further improvement of the functionality of the device according to the invention.

It is preferred if the growth factors are selected from the group consisting of: Platelet Derived Growth Factor” (PDGF), “Vascular Endothelial Growth Factor” (VEGF), “Colony Stimulating Factor” (CSF), “Epidermal Growth Factor” (EGF), “Nerve Growth Factor” (NGF), “Fibroblast Growth Factor” (FGF) and/or growth factors of the “Transforming Growth Factor” (TGF) superfamily. Growth factors of the group of the TGF superfamily are e.g. BMPs (bone morphogenetic proteins) such as BMP-2 and BMP-7.

This measure has the advantage that appropriate growth factors are already provided. In the case of the use of the BMP a differentiation of the bound MSCs into osteocytes affects the promotion of the adherence of the bone substitution or replacement implant according to the invention.

The invention also relates to a method for the production of a device comprising at least one surface which comes into contact with biological tissue and/or liquid, which is at least partially coated with a substance which promotes the binding of mesenchymal stem cells (MSCs), comprising the following steps: (1) providing nucleic acid molecules, and (2) binding the nucleic acid molecules of step (1) to the surface of a device, whereby the nucleic acid molecules comprise the before-described aptamers.

It shall be understood that the before-mentioned features and the features to be explained in the following cannot only be used in the combination indicated in each case, but also in other combinations or in an isolated manner, without departing from the scope of the present invention.

The invention is now explained in detail by means of embodiments which are purely illustrative and do not limit the scope of the invention. This results in further features and advantages of the invention. Reference is made to the enclosed figures.

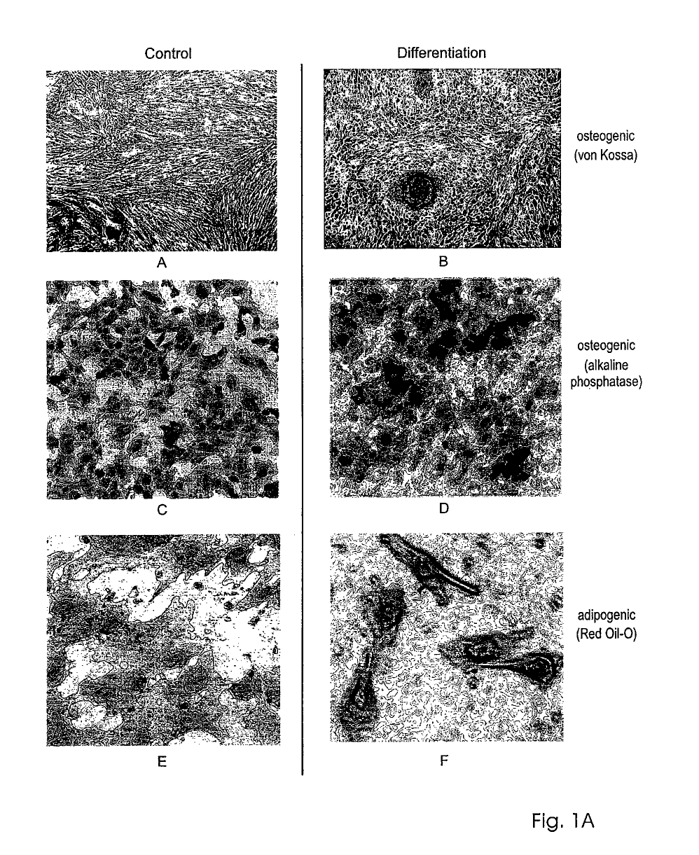

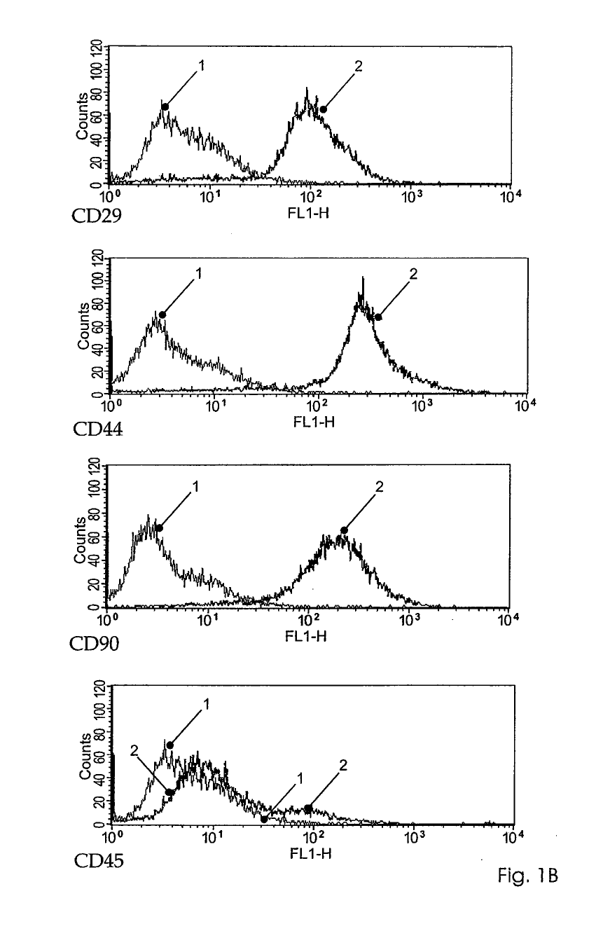

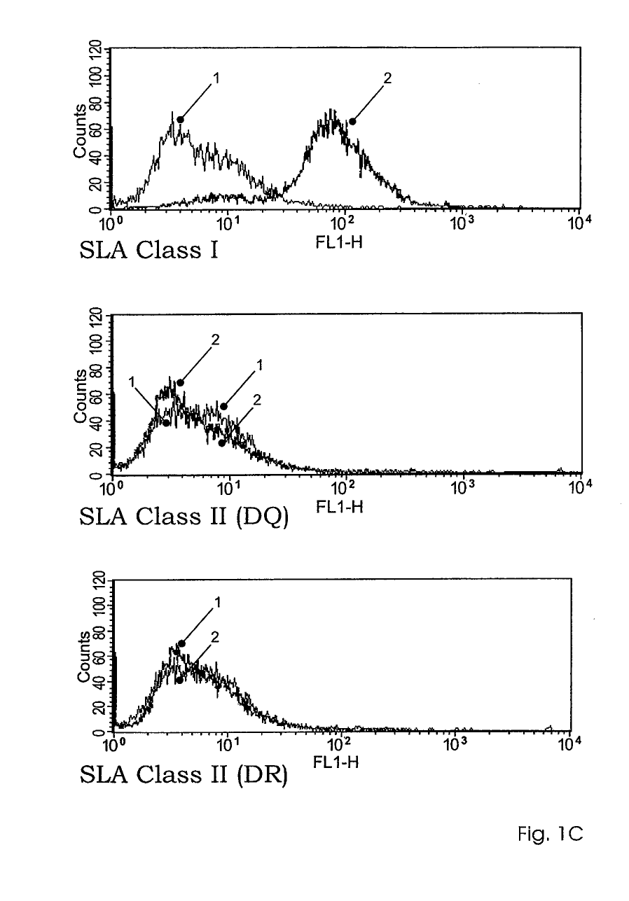

FIG. 1 shows in partial figure (A) the characterization and identification of adult MSCss (aMSCs); ‘AB’ shows an osteogenic staining of aMSC according to Von Kossa in 100-fold magnification, ‘A’ shows the control: ‘CD’ shows a staining for osteogenic alkaline phosphatase and hematoxylin of aMSCs in 200-fold magnification, ‘C’ is the control; ‘EF’ is an adipogenic staining of aMSCs with red oil and hematoxylin in 400-fold magnification, ‘E’ is the control. Partial figure (B) shows the epitope identification of aMSC. The adult porcine aMSC is CD29⁺, CD44⁺, CD90⁺, SL-class I⁺, SLA-class II DQ⁻, SLA-class II DR⁻(the curve 1 is the isotype control).

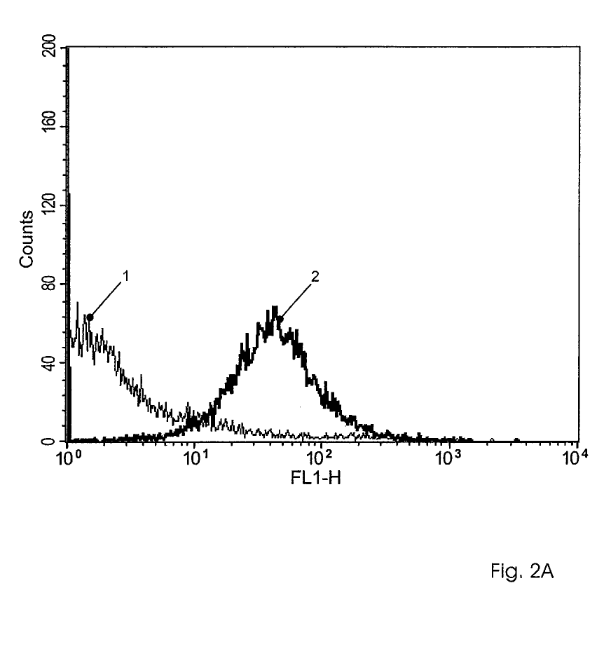

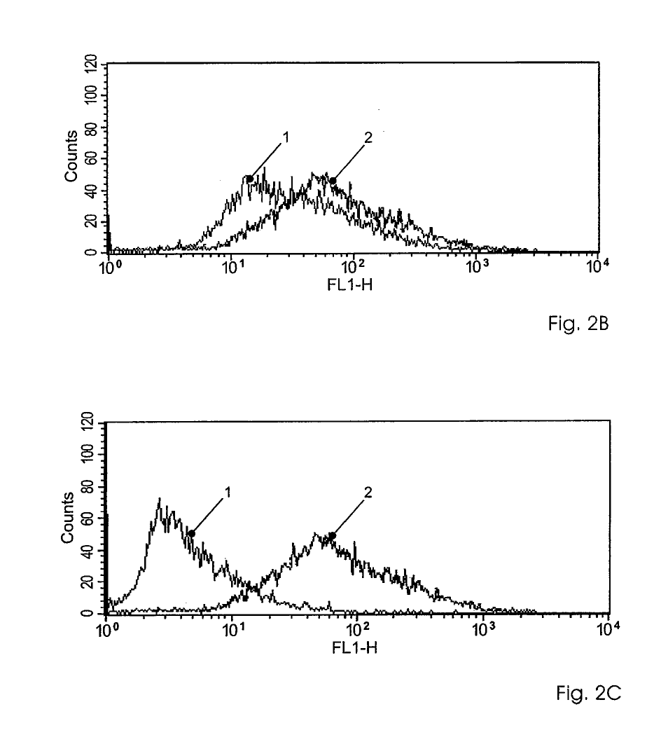

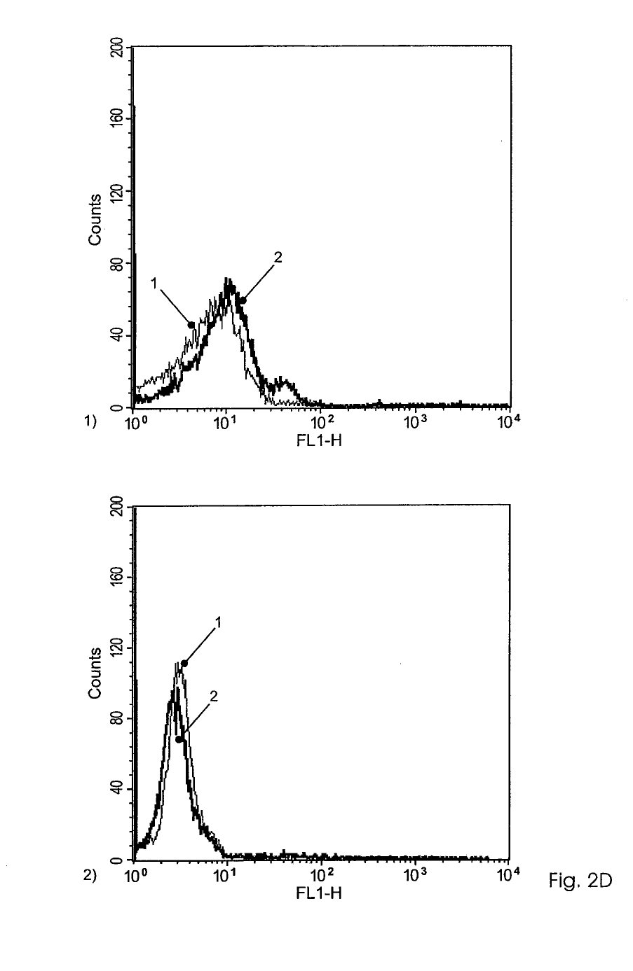

FIG. 2 shows the binding of a selected aptamer (G-8) to aMSCs by means of FACS; in partial figure (A) the curve 2 is the porcine aMSCs incubated with FITC-G-8, the curve 1 is the murine P19-cells incubated with FITC-G-8. In partial figure (B) the curve 2 shows porcine aMSC incubated with FITC-G-8, the curve 1 is the rat aMSCs incubated with FITC-G-8. In partial figure (C) the curve 2 shows porcine aMSCs incubated with FITC-G-8, the curve 1 shows human aMSCs incubated with FITC-G-8. Partial figure (B) shows whole bone marrow FACS assay. Partial FIG. 1 shows the binding of the aptamer G-8 to bone marrow, the partial FIG. 2 shows the binding of the aptamer G-8 with peripheral blood (the curve 1 shows the aptamer G-8 incubated with cells; the curve 2 shows the cell control).

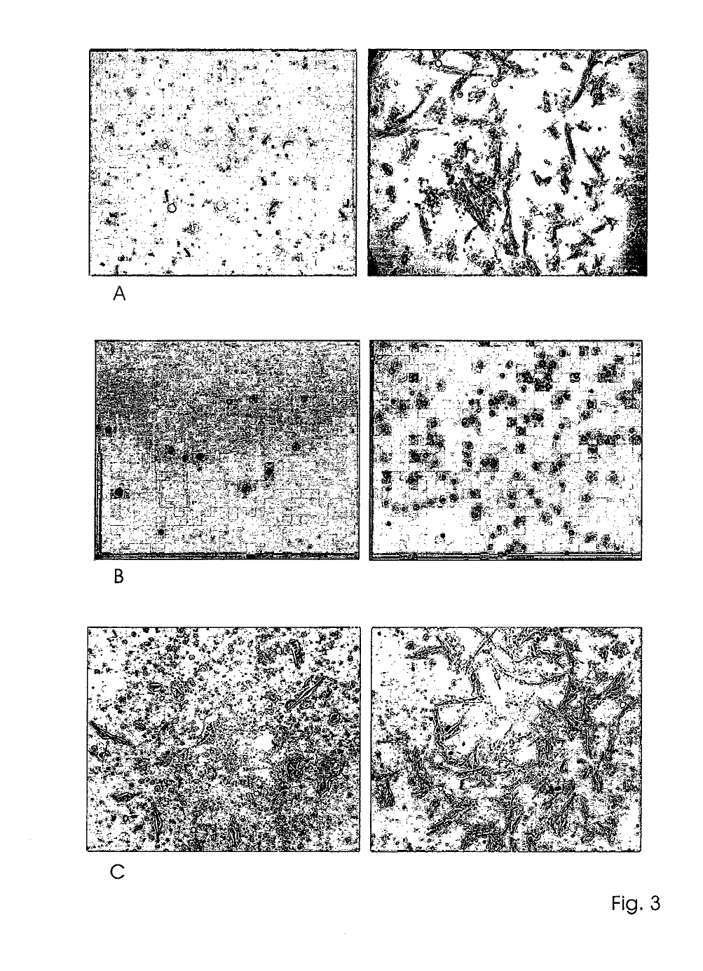

FIG. 3 shows in the partial figure (A) the aptamer-based cell sorting. The cells which bind to the biotinylated aptamer can be pulled down together with anti-biotin microbeads (right) and grow well on culture flasks, while the pure microbeads do not bind to the cells. The cells were washed through the magnetic filter and no cells were held on the magnetic columns, resulting in a fewer amount of cells on the culture flasks (left) (×100). The partial figure (B) shows the surface binding of aMSCs to aptamer coated plates. After one hour of incubation the aptamer coated culture plate captured a lot of aMSCs (right); the culture plate coated with the library captured only very few aMSCs (left) (×100). The partial figure (C) shows aMSC captured from bone marrow. The left picture is the control, only beads incubated with whole bone marrow, where there were only very few cells growing on the culture flask. The right picture shows whole bone marrow incubated with the aptamer (fixed on magnetic microbeads), there are more cells congregated and growing (×100).

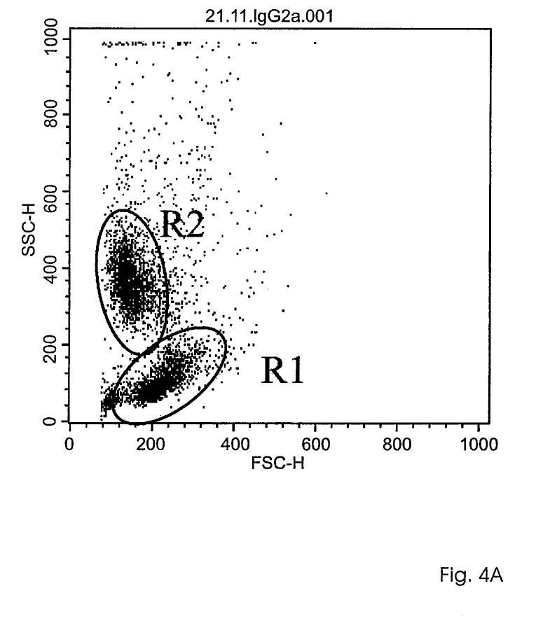

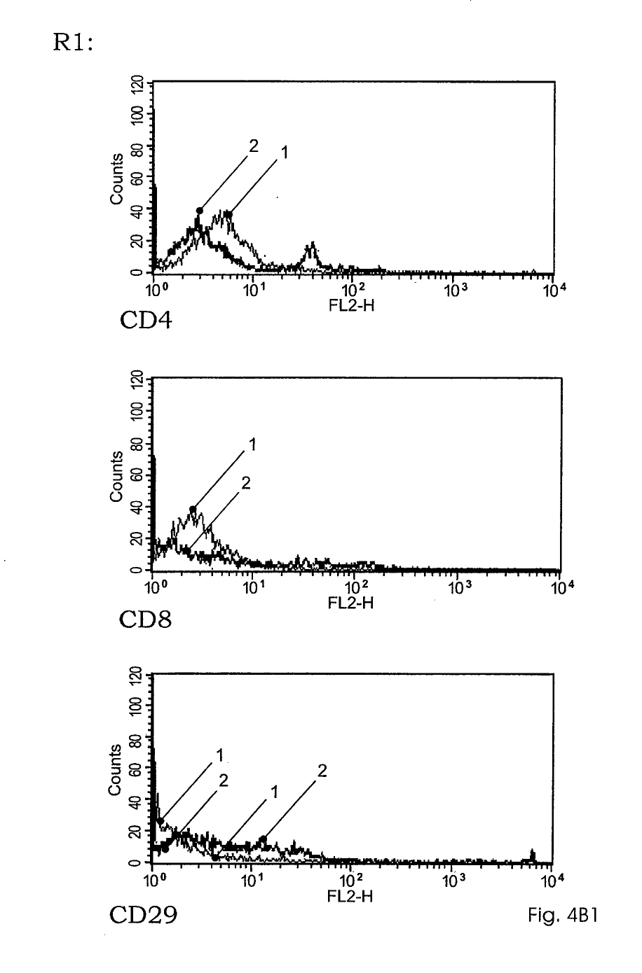

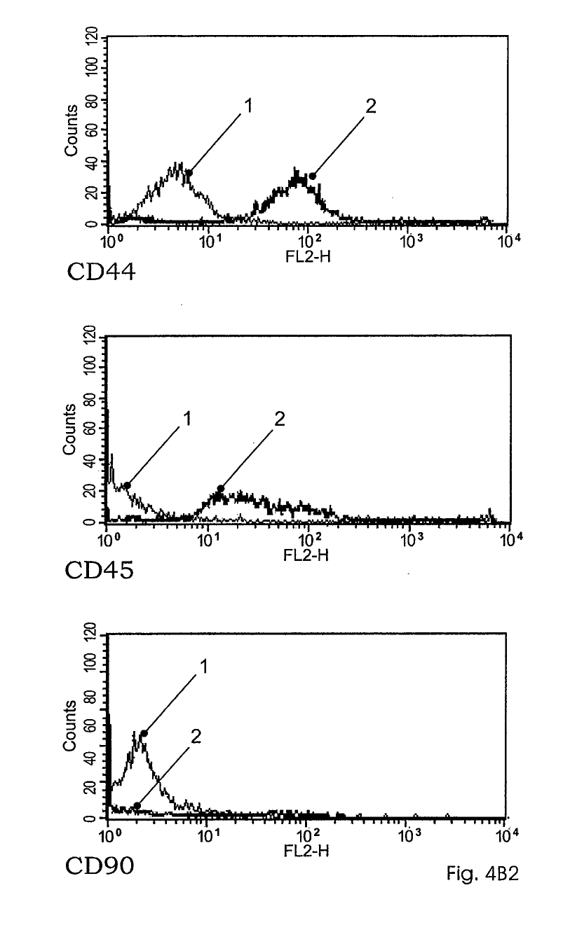

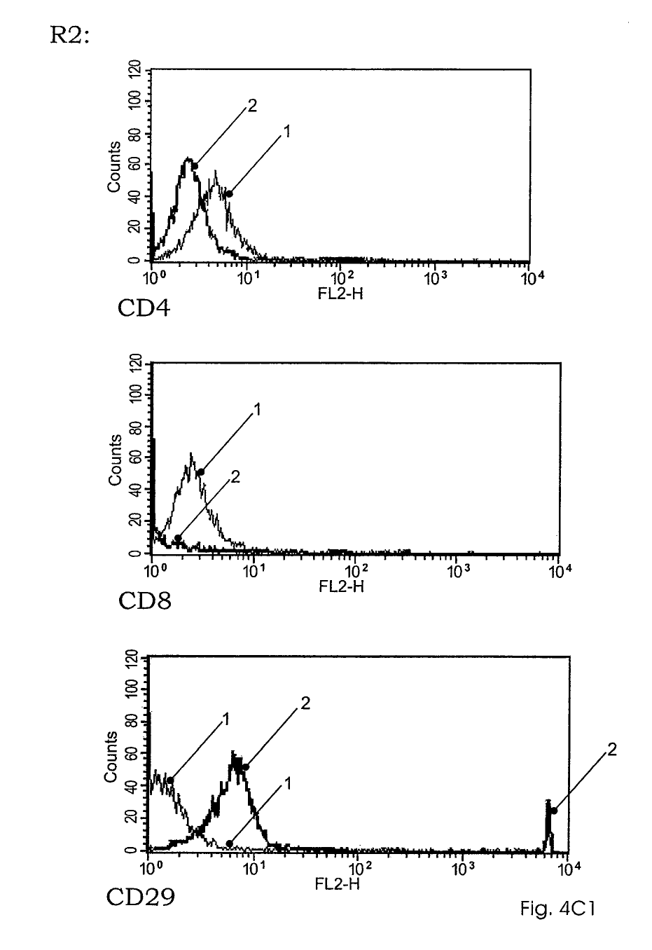

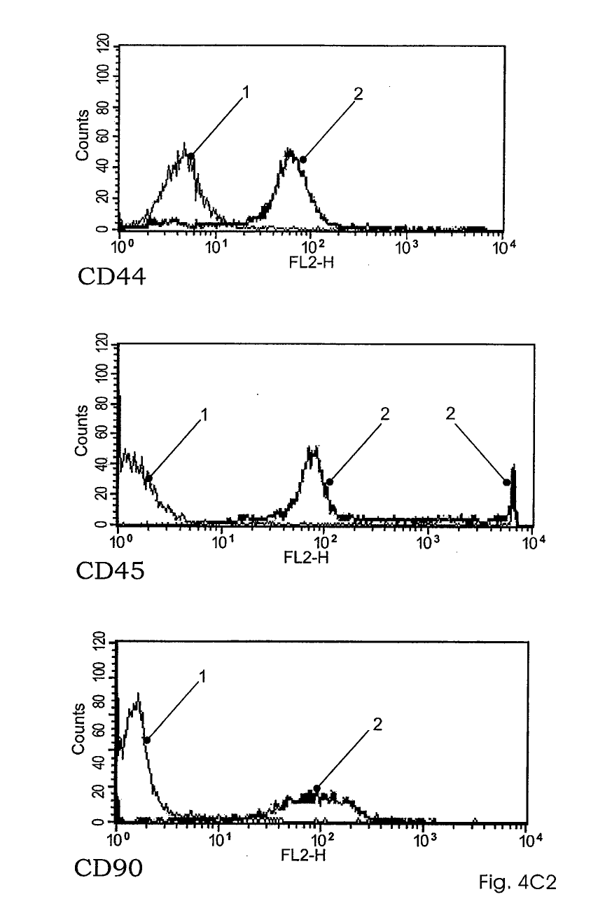

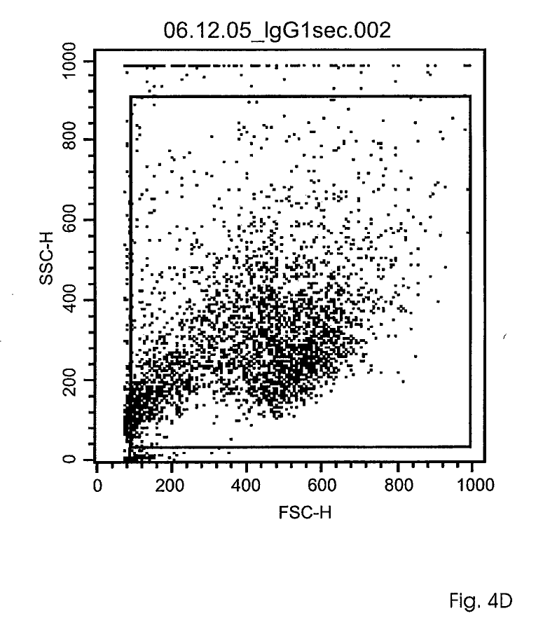

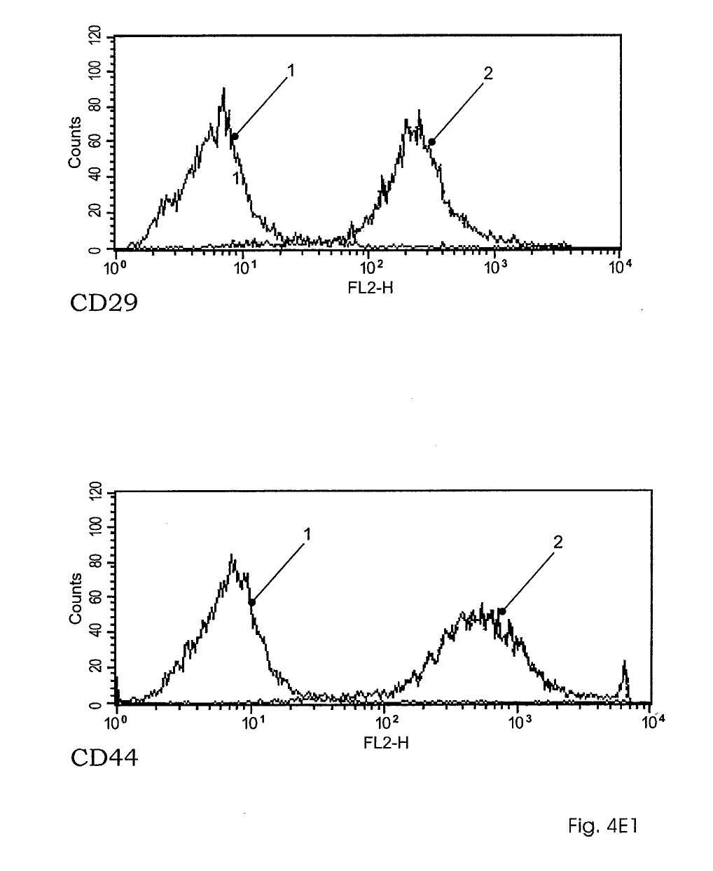

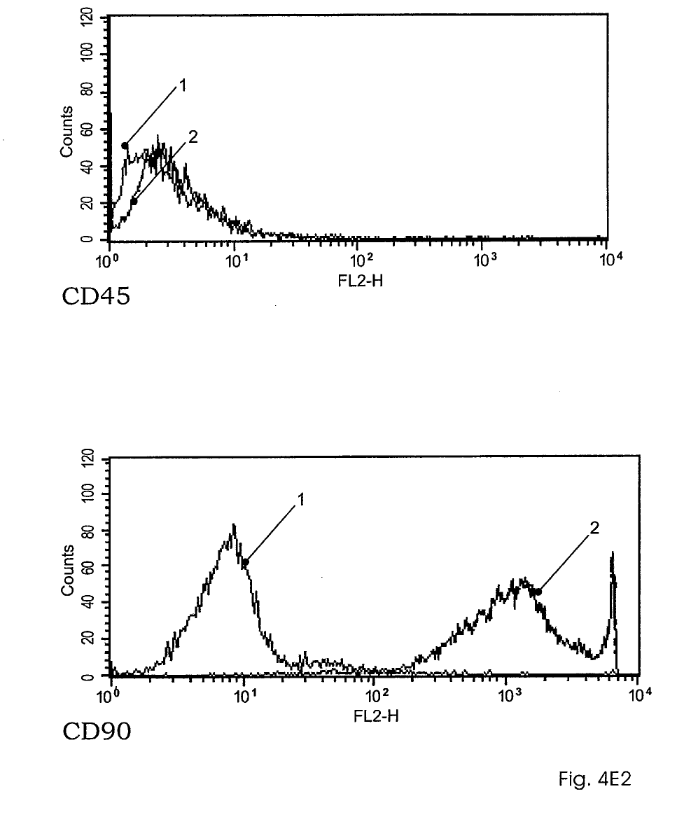

FIG. 4 shows the phenotype identification of the isolated aMSCs. Partial figure (A) shows the subpopulation R1 of the isolated aMSCs, stained with PE labeled antibodies immediately after the isolation. The results shown there were CD4⁻, CD8⁻, CD29⁻, CD44⁺, CD90⁻; the subpopulation R2 of the isolated aMSCs were stained with PE labeled antibodies immediately after the isolation. The results showed CD4⁻, CD8⁻, CD29⁻, CD44⁺, CD90⁺. The curve 1 is the isotype control. Partial figure (B) shows that after two weeks in culture the isolated aMSCs were stained with PE labeled antibodies. The cells were CD29⁺, CD44⁺, CD45⁻, and CD90⁺, the curve 1 is the isotype control.

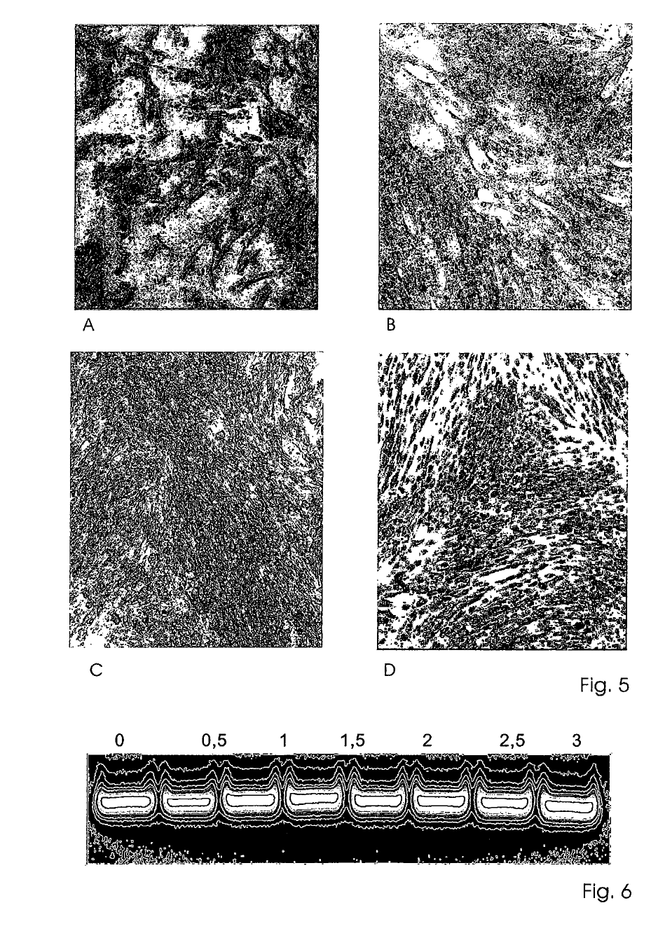

FIG. 5 shows the adipogenic and osteogenic differentiation of the aptamer isolated porcine aMSCs passage 0: (A) adipogenic differentiation after 14 days treatment with hydrocortisone, isobutyl methyl xanthine and indomethacin. Staining with red oil O, hematoxyline counterstaining (×100). (B) control (normal medium; staining with oil red O, hematoxyline counterstaining (×100)). (C) Osteogenic differentiation after 14 days treatment with dexamethason, ascorbic acid and β-glycerol phosphate. Staining for alkaline phosphatase, hematoxyline counterstaining (×100). (D) Control (normal medium; staining for alkaline phosphatase, hematoxyline counterstaining (×100)).

FIG. 6 shows the plasma stability. Analysis of the stability of the aptamer G-8 in human blood plasma by agarose gel electrophoresis. Samples were taken out at different time points from 0 hours to 6 hours. The result shows that the aptamer can resist against degradation until 6 hours at least.

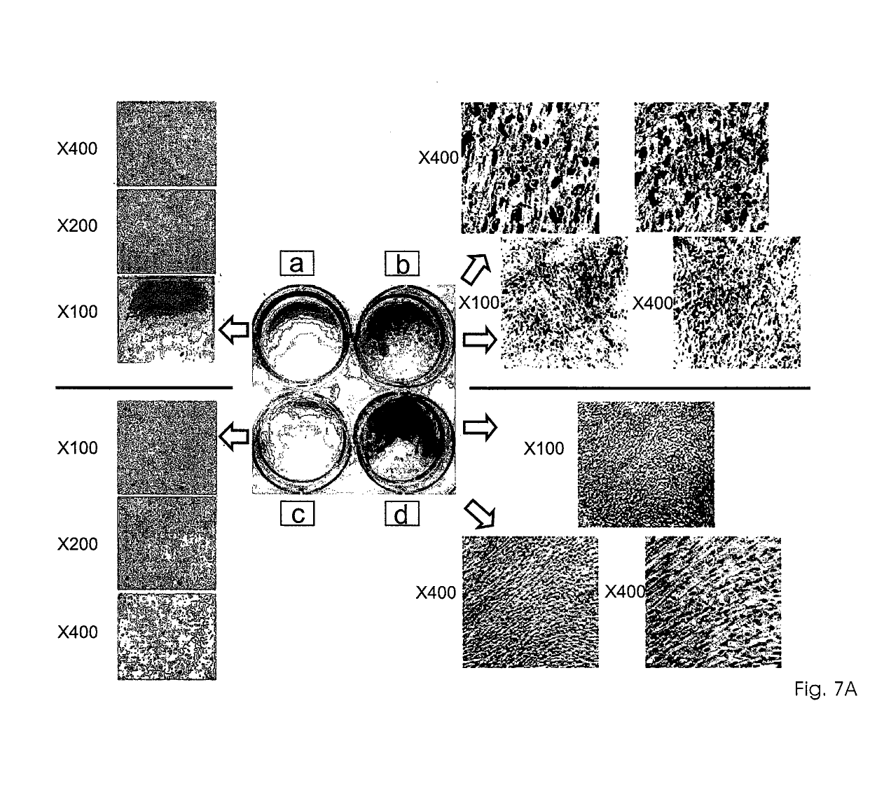

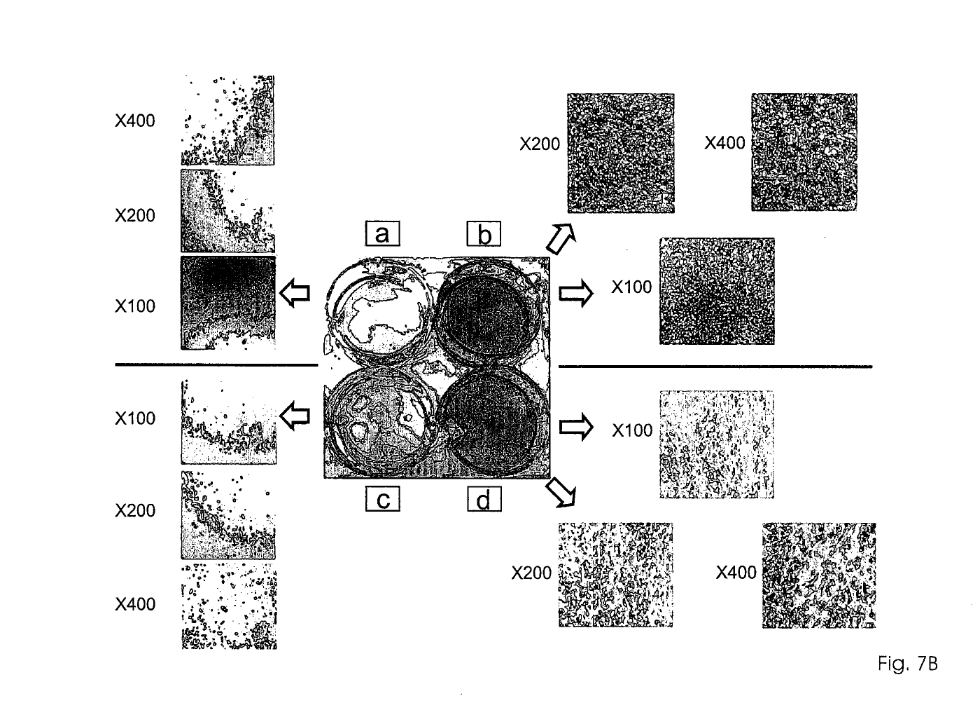

FIG. 7 shows the adipogenic (A) and osteogenic (B) differentiation of the aptamer isolated porcine aMSCs (passage 0) versus plastic adherence procedure for isolation of aMSCs (passage 0). Mononuclear cells were isolated from fresh whole bone marrow by density gradient centrifugation and plated at a density of 500 cells/well (a+c). After 24 hours the medium was changed to remove non-adherent cells. Then, adipogenic or osteogenic or normal medium was added. Aptamer isolated aMSCs were plated at a density of 500 cells/well (b+d). After 24 hours the medium was changed and adipogenic or osteogenic or normal medium was added. After five weeks, when the aptamer sorted cells reached confluency, the staining was started: (A) (adipogenic differentiation): a: whole bone marrow—24 hours adherence, adipogenic medium; b: aptamer isolated aMSCs—24 hours adherence, adipogenic medium; c: whole bone marrow—24 hours adherence, control (normal medium); d: aptamer isolated aMSCs—24 hours adherence, control (normal medium). Staining with red oil O, hematoxyline counterstaining. (B) (osteogenic differentiation): a: whole bone marrow—24 hours adherence, osteogenic medium; b: aptamer isolated aMSCs—24 hours adherence, osteogenic medium; c: whole bone marrow—24 hours adherence, control (normal medium); d: aptamer isolated aMSCs—24 hours adherence, control (normal medium). Staining for alkaline phosphatase, hematoxyline counterstaining. No cell growth could be detected in the wells a and c (plastic adherence procedure for the isolation of aMSCs), whereas aptamer isolated cells (b and d) grew well and showed adipogenic (A, b) and osteogenic (B, b) differentiation.

### DESCRIPTION OF PREFERRED EMBODIMENTS

### 1. Material and Methods

1.1 aMSCs Isolation and Cultivation

Fresh bone marrow was extracted from porcine femur under sterile conditions. The animals (pigs, German landrace, 50 kg, male) were kept and treated according to the Animal Control Instructions of the University of Tübingen. Porcine aMSCs were isolated according to known modification methods; cf. Ponomarev et al. (2003), Preliminary results of enhanced osteogenesie by Fibrogammin and mesenchymal stem cells on chronOS cylinders, European Cells and Materials 5, page 80. Briefly, mononuclear cells (MNCs) were isolated from bone marrow aspirate by centrifugation over Ficoll Hispopaque Layer (30 min, 300 g, density 1.077). After the centrifugation, the cells were cultivated under standard culture conditions with low-glucose Dulbecco's modified Eagle's medium (DMAM; Gibcol) supplemented with 10% fetal calf serum, penicillin (50 U/ml), and streptomycin (50 μg/ml). The medium was changed after 24 hours and then twice a week. When the cells reached 80% confluence they were detached by 0.25% trypsin EDTA solution and replated for the preparation of SELEX and differentiation potential assessments.

The rat and human aMSCs for the specificity tests (FACS with aptamer) were isolated and characterized in the same way. The animals Spraque Dawley rats) were kept and treated according to the Animal Control Instructions of the University of Tübingen. The human bone marrow was taken in the course of orthopaedical operations and approved by the local committee of ethics of the University of Tübingen according to the Declaration of Helsinki. The murine P19 cells were purchased from ATCC (Manassas, Va., United States of America).

1.2 aMSC Characteristics

The potential of aMSCs to differentiate into adipogenic and osteogenic lineages was assayed as follows. For the osteogenic differentiation, the aMSCs were cultured in an osteogenic culture medium which contained 0.2 mM L-ascorbic acid, 2-phosphate magnesium salt, n-hydrate, and 0.01 mM dexamethason (Dex) (Sigma-Aldrich Co.), 10 mM β-glycerol phosphate. After 21 days, the sub-cultured cell layers were washed with phosphate buffered saline PBS and fixed with 4% paraformaldehyde and stained according to the alkaline phosphatase staining kit (Sigma kit #85). After five weeks of being sub-cultured, the deposition of mineralized bone matrix was identified by Von Kossa staining. Cell layers were fixed with 4% paraformaldehyde, incubated with 2% silver nitrate solution (w/v) for 10 minutes in the dark, washed thoroughly with deionized water and then exposed to UV light for 15 minutes. For the adipogenic differentiation, aMSCs were stimulated with growth medium supplemented with 0.5 mM hydrocortisone, 0.5 mM 3-isobutyl-1-methyl xanthine and 60 μM indomethacine (Sigma-Aldrich) for three weeks with the medium change of twice a week. The cells were washed twice with PBS, fixed with 10% formalin for 10 minutes, washed with distilled water, rinsed in 60% isopropanol and covered with a 0.3% red oil O solution (Sigma-Aldrich) in 60% isopropanol. After 10 minutes, cultures were briefly rinsed in 60% isopropanol and thoroughly washed in distilled water and left to dry at room temperature. The surface marker identification with the cultured MSCs was performed by FITC labeled monoclonal antibodies against CD29, CD44, CD45, CD90, SLA-class I, SLA-class DQ and SLA DR (Becton Dickinson, Germany, Heidelberg). For the isotype controls, non-specific mouse IgG was used instead of the primary antibody.

1.3 Selection of the Aptamer Binding to aMSCs

### 1.3.1 DNA Library and Primers

The DNA oligonucleotide library contains a 40-base central random sequence flanked by primer sites on either side (for the porcine MSC aptamers: 5′-GAATTCAGTCGGACAGCG-N40-GATGGACGAATATCGTCTCCC-3′; for the human MSC-aptamers: 5′-GGGAGCTCAGAATAAACGCTCAA-N50-TTCGACATGAGGCCCGAAAC-3′). The size of the library is about 10¹⁵. The FITC labeled forward primer (5′-C₁₂—FITC-GAATTCAGTCGGACAGCG-3′ and the biotin labeled reverse primer (5′-Bio-GGGAGACGATATTCGTCCATC-3′) were used in the PCR to obtain the double-stranded DNA and to separate the single-stranded DNA by streptavidin coated magnetic beads (M-280-Dynabeads, Dynal, Hamburg, Germany). The library and all primers were synthesized by Operon Technologies (Cologne, Germany).

### 1.3.2 SELEX Procedure

The selection of the DNA aptamers against porcine aMSCs was performed as follows. 4 nmol ssDNA pools were denatured by heating at 80° C. for 10 minutes in a selection buffer containing 50 mM Tris-HCl (pH 7.4), 5 mM KCl, 100 mM NaCl, 1 mM MgCl₂, and 0.1% NaN3 and the renatured at 0° C. for 10 minutes. To reduce background binding, a fivefold molar excess of yeast tRNA (Invitrogen, Karlsruhe, Germany) and bovine serum albumin (BSA, Sigma-Aldrich, Munich, Germany) were added. The mesenchymal stem cells (passage 2, 10⁶ cells for the first round and 10⁵ cells for further rounds) were incubated with ssDNA at 37° C. for 30 min in selection buffer. Partitioning of bound and unbound ssDNA sequences was done by centrifugation. After centrifugation and being washed three times with 1 ml selection buffer (0.2% BSA), cell bound ssDNA were amplified by PCR (Master Mix from Promega, Mannheim, Germany). FITC and biotin labeled primers were used in the PCR amplification (25 cycles of 1 min at 94° C., 1 min at 48° C., and 1 min at 72° C., followed by 10 min at 72° C.). For the FACS analysis FITC labeled ssDNA was prepared as described above. Aptamers obtained from the tenth round of selection were PCR amplified using unmodified primers and cloned into Escherichia coli using the TA cloning kit (Invitrogen). Plasmids of individual clones were isolated by the plasmid extraction kit (Qiagen, Dülsseldorf, Germany), and inserts were amplified by PCR and sequenced with the ABI PRISM® 377 DNA Sequencer (Applied Biosystems, Darmstadt, Germany). Individual FITC aptamers were prepared to perform the binding affinity tests.

The selection of DNA aptamers against human aMSCs was performed correspondingly.

### 1.4 Aptamer Binding to MSCs

1.4.1 FACS Assay of Aptamer Binding Affinity to aMSCs

200 pmol of the FITC labeled aptamer were incubated with 10⁵ aMSCs at 37° C. for 30 min, washed three times and analyzed by flow cytometry (BD, Heidelberg, Germany), the same amount of murine P19 cells, rat MSCs incubated with the aptamer were used as a control. The secondary structure of the aptamer was analyzed by DNASYS software (version 2.5; Hitachi Software Engineering Co.).

1.4.2 Aptamer Binding to aMSCs

Biotinylated aptamers were synthesized by OPERON and incubated with 10⁵ aMSCs for 30 min at 37° C., washed three times and incubated with anti-biotin microbeads (Miltenyi Biotec, Bergisch Gladbach, Germany) for 15 min at 0° C. The same number of aMSCs without aptamer was incubated with anti-biotin microbeads and acted a negative control. The mixture was washed three times and filtered through a magnetic column. Then the column was removed from the magnet holder and the beads were put into cell culture medium.

1.4.3 Aptamer Binding to aMSCs in Whole Bone Marrow Blood

FACS: 10 ml fresh bone marrow blood was lysed with ammonium chloride and incubated with FITC labeled aptamer (200 pmol) for 30 min at 37° C. After being washed three times, the cells were analyzed by FACS. The same amount of peripheral blood was treated identically to act as the control.

Capture experiment: 20 ml fresh bone marrow was lysed with ammonium chloride and re-suspended with PBS (2% FBS, 1 mM EDTA). FcR blocking antibody and 1 nmol aptamer were added to the bone marrow solution for 30 min at room temperature. EasySep biotin selection cocktail (cellsystems, St. Katharinen, Germany) was added and incubated for 15 min. Then EasySep magnetic nano particles were added and incubated for 10 min. The mixture was put into the magnet and set aside for 5 min. The supernatant was poured out and the magnetically labeled cells were washed twice with buffer and further cultured.

1.5 Aptamer Mediated aMSCs Adhesion on a Solid Surface

a 12-well cell culture plate (Greiner, Nürtingen, Germany) was coated with Streptavidin over night at 4° C., and then washed with PBS-T (0.05% Tween-20) for several times. The biotinylated aptamer and the biotinylated library (control) (1 nmol) were added to different wells and incubated at 30° C. for 4 hours. The plate was washed with PBS-T and incubated with aMSCs at 37° C. for 30 min with gentle shaking. Then the medium was removed from the plate and the non-adherent cells were discarded. The cell attachment was observed under an inverse microscope (Zeiss Axiovert 135, Zeiss, Oberkochen, Germany).

1.6 FITC Aptamer Mediated aMSC Isolation

20 ml bone marrow was lysed to remove the red blood cells. 4 nmol FITC labeled aptamer was incubated with the bone marrow for 30 min under 37° C., followed by three washing steps and the bone marrow cells were analyzed by FACS. The FITC-positive cells were isolated and collected for further analyses.

1.7 Characterization of the Sorted aMSCs
1.7.1 Phenotype Identification of the Isolated aMSCs

20 ml of whole bone marrow blood from an adult pig were lysed to remove the red blood cells. The FITC labeled aptamer was added and incubated for 30 min at 37° C. After being washed three times, the cells were analyzed under sterile conditions by high speed FACS (FACS-Sort; Becton Dickinson, Heidelberg, Germany) and the FITC positive cells were isolated and collected in PBS. Some of the isolated cells were analyzed the second time by PE-labeled CD4, CD8, CD29, CD44, CD45, and CD90; the rest of the isolated cells was cultured for two weeks and then analyzed by PE-labeled antibodies CD29, CD44, CD45, and CD90 (Becton Dickinson, Heidelberg, Germany).

1.7.2 Differentiation of the Isolated aMSC

The isolated aMSC were cultured in osteogenic culture medium and adipogenic culture medium. The alkaline phosphatase staining and oil red staining were performed as described.

1.8 Comparison of the Efficiency of aMSC Isolation Between Conventional Plastic Adherence and Aptamer Based aMSC Isolation According to the Invention

To evaluate the efficiency of aMSC isolation, adipogenic and osteogenic differentiation potential as well as the amount of isolated cells were compared. Mononuclear cells were isolated from fresh porcine whole bone marrow by density gradient centrifugation and plated at a density of 500 cells/well. After 24 hours the medium was changed to remove non-adherent cells. Then adipogenic or osteogenic or normal medium was added. Aptamer isolated aMSCs were plated at the same density (500 cells/well). After 24 hours the medium was changed and adipogenic or osteogenic or normal medium was added. After 5 weeks, when the aptamer isolated cells reached confluence, the adipogenic and osteogenic staining procedure was started.

### 1.9 Plasma Stability

Fresh human plasma was prepared by centrifugation (3000 g) of whole blood for 20 min. 8 nmol of the aptamer were incubated at 37° C. in a final volume of 0.5 ml of freshly prepared heparinized human plasma. Samples of 50 μl were removed after 0, 0.5, 1, 1.5, 2, 2.5, 3, and 6 hours. The reactions were terminated by adding of 10 μl of loading buffer and subsequent storage on ice. Full-length and digested oligonucleotides were separated on a 2% agarose gel and photodocumented.

### 2. Results

2.1 aMSC Isolation and Characteristics

Porcine and human aMSCs were successfully isolated from bone marrow via gradient centrifugation, expanded in a monolayer culture and evaluated for osteogenic differentiation potential. Spindle bipolar to polygonal fibroblastic cells were observed after 4 days of the first seeding. The cells reached confluence after 12 days of culture. On the first passage the cells showed a uniform monolayer. The aMSCs cultured in osteogenic medium showed ALP-positive and Von Kossa positive (calcium mineral precipitation) after 8 days and 28 days. The aMSC cultured in adipogenic differentiation medium showed red oil staining, while all the controls were negative (FIG. 1(A)). The surface marker staining showed that the attached cells were CD29⁺, CD44⁺, CD45⁻, CD90⁺, SLA-class I⁺, SLA DQ⁻, and SLA DR⁻(FIG. 1(B)).

2.2 Selection of Aptamers With High Affinity to aMSCs

aMSCs derived from porcine and human bone marrow were used as the target for in vitro selection of aptamers from a random pool of DNA molecules. The starting library consisted of 79-mer single-stranded DNA molecules containing randomized 40-oligonucleotide inserts. This library was applied to a number of cultured cells in the same passage, which minimized non-specific interaction. To monitor the enrichment of specific cell-binding aptamers during the selection, SELEX pools of the second and following rounds were analyzed by FACS after the incubation with aMSCs. In each round of the selection, the concentration of competitor DNA was increased to further selection toward a high-affinity and high-specificity aptamer pool. Analysis of fluorescent labeled pools in successive cycles of selection showed a shift from the second round histogram toward higher fluorescent intensity. After 10 rounds of selection, the fluorescence of the pool showed no further increase, the pool was then cloned and sequenced.

Sequences from 20 clones were obtained, and their inserts were analyzed and sorted into putative families by the alignment of consensus motifs. The motifs were identified by inspection with the aid of computer-assisted search engines. The following table 1 shows the nucleotide sequences of the 20 aptamers which were either obtained via the selection against porcine MSCs or human MSCs, and are specific for MSCs.

TABLE 1
Sequence specific aptamers against MSC

| | | |
|---|---|---|
| SEQ | | MSCs- |
| ID | | origin |
| NO: | Nucleotide sequence (from 5′ to 3′) | (SELEX) |

| | | |
|---|---|---|
| 1 | GAATTCAGTCGGACAGCGCGACTTCGGTTATTACGTTG | pig |
| | TTGGCCTCACAAGGACGCCCGATGGACGAATATCGTCT | |
| | CCC | |
| 2 | GAATTCAGTCGGACAGCGCACGATCCAGATGTCATAGT | pig |
| | TTAGGCTCTCTCTACTACTGATGGACGAATATCGTCTC | |
| | CC | |
| 3 | GAATTCAGTCGGACAGCGGGCGGGAGGTCACGTTGAGA | pig |
| | ATTTACGAGGCAGGGGGCACGATGGACGAATATCGTCT | |
| | CCC | |
| 4 | GAATTCAGTCGGACAGCGGAGGGGCCGCCAAAGCTAGC | pig |
| | TCAAGTGATATCCTGTACTGATGGACCAATATCGTCTC | |
| | CC | |
| 5 | GAATTCAGTCGGACAGCGCACCCGTATGCCAAGTCAGA | pig |
| | TCCAGTGTAGATGCGCGCCCCGATGGACGAATATCGTC | |
| | TCCC | |
| 6 | GAATTCAGTCGGACAGCGCGACACGCGCACGGTTCTCA | pig |
| | TCAATACTGCCTCGCCGGTACGATGGACGAATATCGTC | |
| | TCCC | |
| 7 | GAATTCAGTCGGACAGCGCAGCATGCAGAGGCGTCAAA | pig |
| | TAACGGGACCTCTCGGACGATGGACGAATATCGTCTCC | |
| | C | |
| 8 | GGGAGCTCAGAATAAACGCTCAAGGGGAGTGGTGGAGA | human |
| | AAGGCTTACAGGGTAGATAAGGTTCAGGTGCTTCGTTC | |
| | GACATGAGGCCCGAAAC | |
| 9 | GGGAGCTCAGAATAAACGCTCAAGGGTCATTGCAGGGT | human |
| | AAGGTTGGATTTATTGATGCCTCGGAGTTGGGTGGTTC | |
| | GACATGAGGCCCGAAAC | |
| 10 | GGGAGCTCAGAATAAACGCTCAAGTAGGCGTTGCCTTA | human |
| | GTTATTGTTTTGAGGTAGAGCAGAGTTTTACTCAGTTC | |
| | GACATGAGGCCCGAAAC | |
| 11 | GGGAGCTCAGAATAAACGCTCAACGAGGTGGATGACAG | human |
| | GGTATGTGGATTGGTAGTGTGTTTGGTGCTAACGCTTC | |
| | GACATGAGGCCCGAAAC | |
| 12 | GGGAGCTCAGAATAAACGCTCAAGGAGGAAGGGTTACG | human |
| | GAGGAAGAGTTAGGATCGGTGGGGATGATGATGGGTTC | |
| | GACATGAGGCCCGAAAC | |
| 13 | GGGAGCTCAGAATAAACGCTCAAGGTTTAATGTGTGGG | human |
| | TAGTTGGGCGTGACGGGGTAGTCCTGGGGGTTAGGTTC | |
| | GACATGAGGCCCGAAAC | |
| 14 | GGGAGCTCAGAATAAACGCTCAAGTGGAGTGGCCGTAG | human |
| | TCTGGCCAGGTCCCGTTGGTGATGGGTAGAGTGGGTTC | |
| | GACATGAGGCCCGAAAC | |
| 15 | GGGAGCTCAGAATAAACGCTCAATTTGCGCTGGATGCG | human |
| | ATAACGTGTTCGACATGAGGCCCGGATCCACTCCCTTC | |
| | GACATGAGGCCCGAAAC | |
| 16 | GGGAGCTCAGAATAAACGCTCAATGTGCTTATGCTCGA | human |
| | GATGGTGTTATCCGTGTTGCCACGATGGGGGGACCTTC | |
| | GACATGAGGCCCGGATC | |
| 17 | GGGAGCTCAGAATAAACGCTCAATGGATGGGTGGGCGT | human |
| | AGGTGAGGTGTTGTAAGAGCCTCTCCACAGGTGCGTTC | |
| | GACATGAGGCCCGAAAC | |
| 18 | GGGAGCTCAGAATAAACGCTCAATGCTCCAAGGGACAG | human |
| | GGCAAGGGATCTATCCTGCCGCGGGGATGTAAGGCTTC | |
| | GACATGAGGCCCGAAAC | |
| 19 | GGGAGCTCAGAATAAACGCTCAATGGGGGQAAGCGGAC | human |
| | TGTTCGCACTTAGGGCGTATGATGGTAGTGGACCGTTC | |
| | GACATGAGGCCCGAAAC | |
| 20 | GGGAGCTCAGAATAAACGCTCAAGAGTAATGTAGGGTG | human |
| | AAGGGTGTGGGGGCTATGGGGATAGTGGCACGGCCTTC | |
| | GACATGAGGCCCGAAAC | |

2.3 Binding of Aptamers to aMSCs

FACAS-tests: The fluorescence of a binding of an exemplary aptamer comprising the nucleotide sequence SEQ ID NO: 6 (G-8) to an aMSC is shown in FIG. 2(A) to 2(C), which showed the specific binding of the aptamers to aMSCs.

Isolation experiment: aMSCs which bound to the biotinylated aptamer could be isolated and congregated using anti-biotin microbeads. When filtered through a magnetic column, aMSCs could be fixed by the biotinylated aptamer. As shown in FIG. 3(A), the anti-biotin microbeads alone (“microbeads”) could not isolate aMSCs, so there are no cells growing in the culture flask (left image, negative control). The anti-biotin microbeads with a biotinylated aptamer fixed on the surface can bind to aMSCs, therefore growing cells could be detected (right image). This result shows that the aptamer is able to isolate aMSCs from the cell solution.

2.4 Binding of Aptamers to aMSCs in Whole Bone Marrow

FACS assay: The aptamer G-8 shows almost no binding to peripheral blood cells compared to the whole bone marrow (FIG. 1(B)).

Capture experiment: With the EasySep biotin selection kit aMSCs from whole bone marrow could be labeled with a biotinylated aptamer and isolated directly. As shown in FIG. 3(B) left, there was no specific binding between the beads and aMSCs, resulting in few cells growing on the culture plate. The right image demonstrates that aMSCs can be captured on the aptamer labeled beads and can grow well on culture plates (FIG. 3(B)).

2.5 Aptamer Mediated aMSC Adhesion on a Solid Surface

The biotinylated aptamer was immobilized onto a streptavidin coated plate followed by aMSCs flow over the surface. Compared to the plate without coated aptamer, the plate with aptamer coating attached more cells in a short time. The result shows that the aptamer could bind with the target well when being immobilized on a solid surface (FIG. 3(C)).

2.6 Characterization of the Isolated aMSCs
2.61 Phenotypic Identification of the Isolated aMSCs

Mononuclear cells from bone marrow were collected with the FITC labeled aptamer G-8 by high-speed-FACS and analyzed by PE-labeled antibodies. The result shows two subpopulations of isolated cells. The first subpopulation (R1) containing small granular cells was CD4⁻ (82.2%), CD8⁻ (80.5%), CD29⁻ (70.7%), CD44⁺ (90.9%), CD45⁺ (86.4%), and CD90⁻ (77.6%). The second subpopulation (R2) containing small and densely granular cells was CD4⁻ (98.9), CD8⁻ (98.9%), CD29⁻ (83.7%), CD44⁺ (87.7%), CD45⁺ (99.2%), and CD90⁺ (91.8%). The isolated cells were cultured for 14 days (passage 0) and also stained by PE-labeled antibodies. The results showed that they were CD29⁺ (98.0), CD44⁺ (99.6%), CD90⁺ (99.5), and CD45⁻ (87.6%) which are accordant with previously described markers of aMSCs in culture (FIG. 4). In contrast to the freshly sorted cells no distinct subpopulation could be detected and the cultured cells upregulated CD29 and lost the CD45 antigen.

2.6.2 Differentiation of the Isolated aMSCs

The adipogenic and osteogenic differentiation of the aptamer-isolated porcine aMSCs in passage 0 showed that the isolated aMSCs have a high potential to differentiate into adipocytes and osteoblasts (FIG. 5).

2.7 Efficiency of the aMSC Isolation

No cell growth could be detected in wells, in which mononuclear cells from whole bone marrow were seeded (initially plated: 500 cells/well; conventional 24 hour plastic adherence procedure for isolation of aMSCs, FIG. 7(A)a; FIG. 7(B)c), whereas aptamer-isolated cells grew well and showed adipogenic (FIG. 7(A)b) and osteogenic (FIG. 7(B)b) differentiated (initially plated: 500 cells/well; medium change after 24 hours).

This result demonstrates that the method according to the invention for the isolation of MSCs is clearly superior to the up to now performed method of the art where the isolation of the MSCs occurs via plastic adherence.

2.8 Plasma Stability

For clinical or therapeutical applications, the aptamers should be resistant against rapid degradation by exo- and endonucleases. Human plasma predominantly contains a high 3′-exonuclease activity. In human blood plasma, the unmodified aptamer G-8 resists to the degradation of nucleases for 6 hours which was detected by agarose gel analysis (FIG. 6) and does not need extra modification to improve the stability.

1. A device comprising at least one surface which comes into contact with biological tissue and/or liquid, which is at least partially coated with a substance which mediates the binding of mesenchymal stem cells (MSCs), wherein the substance is an aptamer.

2. The device of claim 1, wherein the aptamer is a nucleic acid molecule, which comprises at least one of the sequences SEQ ID NO: 1 to SEQ ID NO: 20.

3. The device of claim 1, wherein the device is an implant.

4. The device of claim 1, wherein the surface comprises a material which is selected from the group consisting of: polytetrafluoroethylene, polystyrene, polyurethane, polyester, polylactid, polyglycolic acid, polysulphone, polypropylene, polyethylene, polycarbonate, poly-vinyl chloride, polyvinyl difluoride, polymethyl methacrylate, hypoxylapatite, isopropyl-acrylamide, texin or copolymers thereof, nylon, silanized glass, ceramics, metals, in particular titanium, and mixtures thereof.

5. The device of claim 1, wherein the aptamers are directly and/or via linker molecules attached to said one surface.

6. The device of claim 5, wherein the linker molecule is N-succinimidyl-3-(2-pyridyl-dithio)propionate.

7. The device of claim 1 additionally comprising growth factors.

8. The device of claim 7, wherein the growth factors are selected from the group consisting of: Platelet Derived Growth Factor” (PDGF), “Vascular Endothelial Growth Factor” (VEGF), “Colony Stimulating Factor” (CSF), “Epidermal Growth Factor” (EGF), “Nerve Growth Factor” (NGF), “Fibroblast Growth Factor” (FGF) and growth factors of the “Transforming Growth Factor” (TGF) superfamily.

9. A method for the isolation of mesenchymal stem cells (MSCs) from biological tissue and/or liquid, comprising the following steps:
(1) providing biological tissue containing MSCs and/or biological liquid containing MSCs,
(2) contacting said tissue and/or said liquid with a substance which binds to MSCs,
(3) incubating for a period of time which is sufficient for the binding of the MSCs to the substance, and
(4) isolating the MSCs which are bound to the substance, wherein the substance is an aptamer.

10. The method of claim 9, wherein the aptamer is a nucleic acid molecule which comprises at least one of the sequences SEQ ID NO: 1 to SEQ ID NO: 20.

11. The method of claim 9, wherein the aptamer comprises a detectable and/or selectable marker.

12. The method of claim 9, which it is performed within the frame of a fluorescence activated cell sorting (FACS) and/or magnetic cell sorting (MACS).

13. A nucleic acid molecule, which is designed in such a manner that it selectively and highly specifically binds to mesenchymal stem cells (MSCs).

14. The nucleic acid molecule of claim 13 comprising at least one of the sequences SEQ ID NO: 1 to SEQ ID NO: 20.

15. The nucleic acid molecule of claim 13, comprising a detectable and/or selectable marker.

16. A method for the production of a device comprising at least one surface which comes into contact with biological tissue and/or liquid, which is at least partially coated with a substance which mediates the binding of mesenchymal stem cells (MSCs), comprising the following steps:
(1) providing nucleic acid molecules, and
(2) binding the nucleic acid molecules of step (1) to the surface of a device, wherein said nucleic acid molecules comprise the nucleic acid molecule of claim 13.


SEQUENCE LISTING (24 sequences)

SEQ ID NO: 1 - DNA, 79 bp, Artificial sequence
  Synthetic oligonucleotide aptamers
gaattcagtcggacagcgcgacttcggttattacgttgttggcctcaacaggacgcccgatggacgaatatcgtctccc

SEQ ID NO: 2 - DNA, 78 bp, Artificial sequence
  Synthetic oligonucleotide aptamers
gaattcagtcggacagcgcacgatccagatgtcatagtttaggctctctctactactgatggacgaatatcgtctccc

SEQ ID NO: 3 - DNA, 79 bp, Artificial sequence
  Synthetic oligonucleotide aptamers
gaattcagtcggacagcgggcgggaggtcacgttgagaatttacgaggcagggggcacgatggacgaatatcgtctccc

SEQ ID NO: 4 - DNA, 78 bp, Artificial sequence
  Synthetic oligonucleotide aptamers
gaattcagtcggacagcggaggggccgccaaagctagctcaagtgatatcctgtactgatggacgaatatcgtctccc

SEQ ID NO: 5 - DNA, 80 bp, Artificial sequence
  Synthetic oligonucleotide aptamers
gaattcagtcggacagcgcacccgtatgccaagtcagatccagtgtagatgcgcgccccgatggacgaatatcgtctccc

SEQ ID NO: 6 - DNA, 80 bp, Artificial sequence
  Synthetic oligonucleotide aptamers
gaattcagtcggacagcgcgacacgcgcacggttctcatcaatactgcctcgccggtacgatggacgaatatcgtctccc

SEQ ID NO: 7 - DNA, 77 bp, Artificial sequence
  Synthetic oligonucleotide aptamers
gaattcagtcggacagcgcagcatgcagaggcgtcaaataacgggacctctcggacgatggacgaatatcgtctccc

SEQ ID NO: 8 - DNA, 93 bp, Artificial sequence
  Synthetic oligonucleotide aptamers
gggagctcagaataaacgctcaaggggagtggtggagaaaggcttacagggtagataaggttcaggtgcttcgttcgacatgaggcccgaaac

SEQ ID NO: 9 - DNA, 93 bp, Artificial sequence
  Synthetic oligonucleotide aptamers
gggagctcagaataaacgctcaagggtcattgcagggtaaggttggatttattgatgcctcggagttgggtggttcgacatgaggcccgaaac

SEQ ID NO: 10 - DNA, 93 bp, Artificial sequence
  Synthetic oligonucleotide aptamers
gggagctcagaataaacgctcaagtaggcgttgccttagttattgttttgaggtagagcagagttttactcagttcgacatgaggcccgaaac

SEQ ID NO: 11 - DNA, 93 bp, Artificial sequence
  Synthetic oligonucleotide aptamers
gggagctcagaataaacgctcaacgaggtggatgacagggtatgtggattggtagtgtgtttggtgctaacgcttcgacatgaggcccgaaac

SEQ ID NO: 12 - DNA, 93 bp, Artificial sequence
  Synthetic oligonucleotide aptamers
gggagctcagaataaacgctcaaggaggaagggttacggaggaagagttaggatcggtggggatgatgatgggttcgacatgaggcccgaaac

SEQ ID NO: 13 - DNA, 93 bp, Artificial sequence
  Synthetic oligonucleotide aptamers
gggagctcagaataaacgctcaaggtttaatgtgtgggtagttgggcgtgacggggtagtcctgggggttaggttcgacatgaggcccgaaac

SEQ ID NO: 14 - DNA, 93 bp, Artificial sequence
  Synthetic oligonucleotide aptamers
gggagctcagaataaacgctcaagtggagtggccgtagtctggccaggtcccgttggtgatgggtagagtgggttcgacatgaggcccgaaac

SEQ ID NO: 15 - DNA, 93 bp, Artificial sequence
  Synthetic oligonucleotide aptamers
gggagctcagaataaacgctcaatttgcgctggatgcgataacgtgttcgacatgaggcccggatccactcccttcgacatgaggcccgaaac

SEQ ID NO: 16 - DNA, 93 bp, Artificial sequence
  Synthetic oligonucleotide aptamers
gggagctcagaataaacgctcaatgtgcttatgctcgagatggtgttatccgtgttgccacgatggggggaccttcgacatgaggcccggatc

SEQ ID NO: 17 - DNA, 93 bp, Artificial sequence
  Synthetic oligonucleotide aptamers
gggagctcagaataaacgctcaatggatgggtgggcgtaggtgaggtgttgtaagagcctctccacaggtgcgttcgacatgaggcccgaaac

SEQ ID NO: 18 - DNA, 93 bp, Artificial sequence
  Synthetic oligonucleotide aptamers
gggagctcagaataaacgctcaatgctccaagggacagggcaagggatctatcctgccgcggggatgtaaggcttcgacatgaggcccgaaac

SEQ ID NO: 19 - DNA, 93 bp, Artificial sequence
  Synthetic oligonucleotide aptamers
gggagctcagaataaacgctcaatggggggaagcggactgttcgcacttagggcgtatgatggtagtggaccgttcgacatgaggcccgaaac

SEQ ID NO: 20 - DNA, 93 bp, Artificial sequence
  Synthetic oligonucleotide aptamers
gggagctcagaataaacgctcaagagtaatgtagggtgaagggtgtgggggctatggggatagtggcacggccttcgacatgaggcccgaaac

SEQ ID NO: 21 - DNA, 79 bp, Artificial Sequence
  Synthetic oligonucleotide primer.
gaattcagtcggacagcgnnnnnnnnnnnnnnnnnnnnnnnnnnnnnnnnnnnnnnnngatggacgaatatcgtctccc

SEQ ID NO: 22 - DNA, 93 bp, Artificial Sequence
  Synthetic oligonucleotide primer.
gggagctcagaataaacgctcaannnnnnnnnnnnnnnnnnnnnnnnnnnnnnnnnnnnnnnnnnnnnnnnnnttcgacatgaggcccgaaac

SEQ ID NO: 23 - DNA, 18 bp, Artificial Sequence
  Synthetic oligonucleotide primer.
gaattcagtcggacagcg

SEQ ID NO: 24 - DNA, 21 bp, Artificial Sequence
  Synthetic oligonucleotide primer.
gggagacgatattcgtccatc